(19) United States
(12) Patent Application Publication
(10) Pub. No.: US 2023/0070561 A1
(43) Pub. Date: Mar. 9, 2023

(54) RADIOSCOPY APPARATUS, RADIOSCOPY METHOD, RADIOSCOPY PROGRAM, FLUOROSCOPIC IMAGE DISPLAY DEVICE, FLUOROSCOPIC IMAGE DISPLAY METHOD, AND FLUOROSCOPIC IMAGE DISPLAY PROGRAM

(71) Applicant: FUJIFILM Corporation, Tokyo (JP)

(72) Inventor: Shin HAMAUZU, Kanagawa-ken (JP)

(73) Assignee: FUJIFILM Corporation, Tokyo (JP)

(21) Appl. No.: 17/819,351

(22) Filed: Aug. 12, 2022

(30) Foreign Application Priority Data
Sep. 3, 2021 (JP) 2021-143922

(51) Int. Cl. A61B 6/00; H04N 5/32; G06T 5/50; A61B 6/12

(52) U.S. Cl. CPC A61B 6/487; H04N 5/32; G06T 5/50; A61B 6/12; A61B 6/542; A61B 6/463; G06T 2207/10121; G06T 2207/20212; G06T 2207/10016; A61B 6/4441

(57) ABSTRACT

A processor performs first fluoroscopy on a subject before a treatment tool is inserted under first fluoroscopy conditions including at least one of a predetermined first tube voltage or a predetermined first tube current to acquire a first fluoroscopic image of the subject. The processor performs second fluoroscopy at a predetermined frame rate on the subject after the treatment tool is inserted under second fluoroscopy conditions including at least one of a second tube voltage higher than the first tube voltage or a second tube current smaller than the first tube current to sequentially acquire a plurality of second fluoroscopic images of the subject.

### CROSS REFERENCE TO RELATED APPLICATIONS

The present application claims priority under 35 U.S.C. § 119 to Japanese Patent Application No. 2021-143922 filed on Sep. 3, 2021. The above application is hereby expressly incorporated by reference, in its entirety, into the present application.

### BACKGROUND

### Technical Field

The present disclosure relates to a radioscopy apparatus, a radioscopy method, a radioscopy program, a fluoroscopic image display device, a fluoroscopic image display method, and a fluoroscopic image display program.

### Related Art

In a surgical operation and a catheter treatment, it is necessary to understand a positional relationship between a treatment tool and a human body structure such as a bone and a blood vessel. However, in the related art, in many cases, the understanding of the positional relationship between the treatment tool and the human body structure relies on the experience and intuition of a doctor, and there are problems of incorrect insertion of the treatment tool and excessive treatment time. Therefore, fluoroscopy that continuously irradiates a subject with radiation from a radiation source during a treatment and displays a fluoroscopic image acquired by the continuous irradiation in real time is performed to understand the positional relationship between the treatment tool and the human body structure.

However, in a case in which the positional relationship between the treatment tool and the human body structure is understood in detail using the fluoroscopic image acquired by the fluoroscopy, the irradiation time of the radiation increases, and the amount of radiation exposure to the subject increases. For this reason, some measures have been taken to protect the subject with a protective plate or to reduce an imaging rate. However, the effect of reducing radiation exposure is not sufficient.

Therefore, a method has been proposed which detects a treatment tool from a fluoroscopic image including the entire subject, irradiates only a region around the treatment tool with radiation to acquire a partial fluoroscopic image, and combines the fluoroscopic image including the entire subject with the partial fluoroscopic image (see JP2014-144053A). According to the method disclosed in JP2014-144053A, since only the region around the treatment tool is irradiated with radiation, it is possible to reduce the amount of radiation exposure to the subject.

However, in the method disclosed in JP2014-144053A, the region around the treatment tool is also irradiated with the same amount of radiation as that in a case in which the fluoroscopic image including the entire subject is acquired. Therefore, it is not possible to reduce the amount of radiation exposure to the region around the treatment tool in the subject.

### SUMMARY OF THE INVENTION

The present disclosure has been made in view of the above circumstances, and an object of the present disclosure is to provide a technique that can further reduce the amount of radiation exposure to a subject in a case in which fluoroscopy is performed.

According to the present disclosure, there is provided a radioscopy apparatus comprising: a radiation source that irradiates a subject with radiation; a radiation detector that detects the radiation transmitted through the subject to generate a fluoroscopic image of the subject; and at least one processor. The processor controls the radiation source and the radiation detector such that first fluoroscopy is performed on the subject before a treatment tool is inserted under first fluoroscopy conditions including at least one of a predetermined first tube voltage or a predetermined first tube current to acquire a first fluoroscopic image of the subject, and controls the radiation source and the radiation detector such that second fluoroscopy is performed at a predetermined frame rate on the subject after the treatment tool is inserted under second fluoroscopy conditions including at least one of a second tube voltage higher than the first tube voltage or a second tube current smaller than the first tube current to sequentially acquire a plurality of second fluoroscopic images of the subject.

“At the predetermined frame rate” means, for example, at a time interval corresponding to a frame rate of a moving image. For example, 25 to 60 frames per second (fps) or the like can be adopted as the predetermined frame rate. As a result, in the present disclosure, the second fluoroscopic images are acquired as a moving image.

In addition, the radioscopy apparatus according to the present disclosure may further comprise an irradiation field stop that regulates a range in which the subject is irradiated with the radiation. The processor may detect the treatment tool from one of the second fluoroscopic images, set the irradiation field stop such that a range, which includes the detected treatment tool and is narrower than that in a case in which the first fluoroscopic image is acquired, is irradiated with the radiation, and regulate the radiation with the set irradiation field stop and irradiate the subject with the radiation to perform the second fluoroscopy after the one second fluoroscopic image is acquired.

Further, in the radioscopy apparatus according to the present disclosure, after acquiring the first fluoroscopic image, the processor may control the radiation source and the radiation detector such that third fluoroscopy which sequentially acquires a third fluoroscopic image at a frame rate lower than the predetermined frame rate is further performed under the first fluoroscopy conditions.

A first fluoroscopic image display device according to the present disclosure comprises at least one processor. The processor acquires the first fluoroscopic image acquired by the radioscopy apparatus according to the present disclosure, sequentially acquires the second fluoroscopic images acquired by the radioscopy apparatus according to the present disclosure, sequentially extracts a region of the treatment tool from each of the second fluoroscopic images, sequentially combines the region of the treatment tool with the first fluoroscopic image to sequentially derive a composite fluoroscopic image at the predetermined frame rate, and sequentially displays the composite fluoroscopic image.

A second fluoroscopic image display device according to the present disclosure comprises at least one processor. The processor acquires the first fluoroscopic image acquired by the radioscopy apparatus according to the present disclosure which performs the third fluoroscopy, sequentially acquires the second fluoroscopic images acquired by the radioscopy apparatus according to the present disclosure which performs the third fluoroscopy, sequentially extracts a region of the treatment tool from each of the second fluoroscopic images, sequentially combines the region of the treatment tool with the first fluoroscopic image to sequentially derive a composite fluoroscopic image at the predetermined frame rate, displays the composite fluoroscopic image, sequentially acquires the third fluoroscopic image acquired by the radioscopy apparatus according to the present disclosure which performs the third fluoroscopy, sequentially combines the region of the treatment tool extracted from the second fluoroscopic image acquired until a next third fluoroscopic image is acquired with the sequentially acquired third fluoroscopic images to sequentially derive other composite fluoroscopic images, and sequentially displays the other composite fluoroscopic images instead of the composite fluoroscopic image.

According to the present disclosure, there is provided a radioscopy method in a radioscopy apparatus including a radiation source that irradiates a subject with radiation and a radiation detector that detects the radiation transmitted through the subject to generate a fluoroscopic image of the subject. The radioscopy method comprises: controlling the radiation source and the radiation detector such that first fluoroscopy is performed on the subject before a treatment tool is inserted under first fluoroscopy conditions including at least one of a predetermined first tube voltage or a predetermined first tube current to acquire a first fluoroscopic image of the subject; and controlling the radiation source and the radiation detector such that second fluoroscopy is performed at a predetermined frame rate on the subject after the treatment tool is inserted under second fluoroscopy conditions including at least one of a second tube voltage higher than the first tube voltage or a second tube current smaller than the first tube current to sequentially acquire a plurality of second fluoroscopic images of the subject.

A first fluoroscopic image display method according to the present disclosure comprises: acquiring the first fluoroscopic image acquired by the radioscopy apparatus according to the present disclosure; sequentially acquiring the second fluoroscopic images acquired by the radioscopy apparatus according to the present disclosure; sequentially extracting a region of the treatment tool from each of the second fluoroscopic images; sequentially combining the region of the treatment tool with the first fluoroscopic image to sequentially derive a composite fluoroscopic image at the predetermined frame rate; and sequentially displaying the composite fluoroscopic image.

A second fluoroscopic image display method according to the present disclosure comprises: acquiring the first fluoroscopic image acquired by the radioscopy apparatus according to the present disclosure which performs the third fluoroscopy; sequentially acquiring the second fluoroscopic images acquired by the radioscopy apparatus according to the present disclosure which performs the third fluoroscopy; sequentially extracting a region of the treatment tool from each of the second fluoroscopic images; sequentially combining the region of the treatment tool with the first fluoroscopic image to sequentially derive a composite fluoroscopic image at the predetermined frame rate; displaying the composite fluoroscopic image; sequentially acquiring the third fluoroscopic image acquired by the radioscopy apparatus according to the present disclosure which performs the third fluoroscopy; sequentially combining the region of the treatment tool extracted from the second fluoroscopic image acquired until a next third fluoroscopic image is acquired with the sequentially acquired third fluoroscopic images to sequentially derive other composite fluoroscopic images; and sequentially displaying the other composite fluoroscopic images instead of the composite fluoroscopic image.

In addition, programs that cause a computer to perform the radioscopy method and the first and second fluoroscopic image display methods according to the present disclosure may be provided.

According to the present disclosure, it is possible to further reduce the amount of radiation exposure to a subject in a case in which fluoroscopy is performed.

### BRIEF DESCRIPTION OF THE DRAWINGS

FIG. 1 is a diagram illustrating a schematic configuration of a radioscopy apparatus to which a fluoroscopic image display device according to a first embodiment of the present disclosure is applied.

FIG. 2 is a diagram illustrating a schematic configuration of the fluoroscopic image display device according to the first embodiment.

FIG. 3 is a diagram illustrating a functional configuration of the radioscopy apparatus and the fluoroscopic image display device according to the first embodiment.

FIG. 4 is a diagram illustrating the timing when a radiation source emits radiation and the timing when a radiation detector detects the radiation in the first embodiment.

FIG. 5 is a diagram illustrating a first fluoroscopic image.

FIG. 6 is a diagram illustrating second fluoroscopic images acquired in the first embodiment.

FIG. 7 is a diagram illustrating an extracted region of a treatment tool.

FIG. 8 is a diagram illustrating composite fluoroscopic images.

FIG. 9 is a diagram illustrating a composite fluoroscopic image display screen.

FIG. 10 is a flowchart illustrating a process performed in the first embodiment.

FIG. 11 is a diagram illustrating the setting of an irradiation field region in a second embodiment.

FIG. 12 is a diagram illustrating second fluoroscopic images acquired in the second embodiment.

FIG. 13 is a flowchart illustrating a process performed in the second embodiment.

FIG. 14 is a diagram illustrating the timing when the radiation source emits radiation and the timing when the radiation detector detects the radiation in a third embodiment.

FIG. 15 is a diagram illustrating other composite fluoroscopic images.

FIG. 16 is a flowchart illustrating a process performed in the third embodiment.

### DETAILED DESCRIPTION

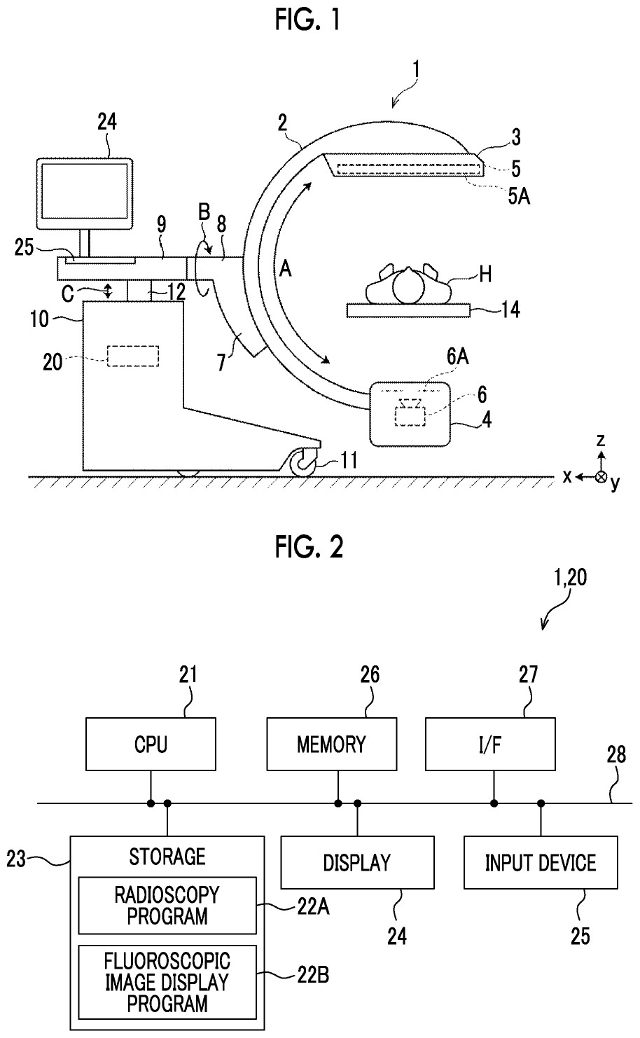

Hereinafter, embodiments of the present disclosure will be described with reference to the drawings. FIG. 1 is a diagram illustrating a schematic configuration of a radioscopy apparatus according to an embodiment of the present disclosure. The radioscopy apparatus according to this embodiment acquires fluoroscopic images of a subject H as a moving image and displays the fluoroscopic images in a case in which a surgical operation, a catheter treatment, or the like is performed on the subject H.

In addition, in this embodiment, it is assumed that the x-axis is set in a left-right direction of FIG. 1, the y-axis is set in a depth direction of FIG. 1, and the z-axis is set in a direction perpendicular to a surface on which the radioscopy apparatus 1 illustrated in FIG. 1 is placed.

As illustrated in FIG. 1, the radioscopy apparatus 1 according to this embodiment comprises a C-arm 2. An imaging unit 3 is attached to one end portion of the C-arm 2, and a radiation emitting unit 4 is attached to the other end portion of the C-arm 2 so as to face the imaging unit 3.

A radiation detector 5, such as a flat panel detector, is provided in the imaging unit 3. In addition, for example, a circuit substrate including a charge amplifier that converts a charge signal read from the radiation detector 5 into a voltage signal, a correlated double sampling circuit that samples the voltage signal output from the charge amplifier, and an analog-to-digital (AD) conversion unit that converts the voltage signal into a digital signal is provided in the imaging unit 3. Further, in this embodiment, in a case in which radiation can be detected and converted into an image, a detector, such as an image intensifier, can also be used.

The radiation detector 5 can repeatedly perform the recording and reading of a radiographic image. A so-called direct-type radiation detector that directly converts radiation, such as X-rays, into charge may be used, or a so-called indirect-type radiation detector that converts radiation into visible light and converts the visible light into a charge signal may be used. In addition, as a method for reading a radiographic image signal, it is desirable to use the following method: a so-called thin film transistor (TFT) reading method which turns on and off a TFT switch to read a radiographic image signal; or a so-called optical reading method which emits reading light to read a radiographic image signal. However, the reading method is not limited thereto, and other methods may be used.

A radiation source 6 is accommodated in the radiation emitting unit 4. Radiation is emitted from the radiation source 6 to the imaging unit 3. The radiation source 6 emits X-rays as the radiation, and an imaging control unit 31, which will be described below, controls the timing when the radiation source 6 emits radiation and the timing when the radiation detector 5 detects the radiation. Further, the imaging control unit 31 controls radiation generation conditions in the radiation source 6, that is, the selection of materials forming a target and a filter, a tube voltage, an irradiation time, and the like.

Furthermore, an irradiation field stop 6A is disposed on an emission side of the radiation source 6. The irradiation field stop 6A has a plurality of shielding plates which can be moved independently and moves the shielding plates in the x direction and the y direction (horizontal direction) in FIG. 1 to change the size of an aperture through which the radiation emitted from the radiation source 6 is transmitted. In addition, the shielding plate is made of a material having a radiation shielding property such as lead. Therefore, the irradiation field stop 6A regulates the range in which the subject H is irradiated with the radiation.

The C-arm 2 according to this embodiment is held by a C-arm holding portion 7 such that it can be moved in the direction of an arrow A illustrated in FIG. 1 and the angle of the imaging unit 3 and the radiation emitting unit 4 with respect to the z direction (vertical direction) illustrated in FIG. 1 can be integrally changed. Further, the C-arm holding portion 7 has a shaft portion 8, and the shaft portion 8 rotatably connects the C-arm 2 to a bearing portion 9. Therefore, the C-arm 2 can be rotated on the shaft portion 8 as a rotation axis in the direction of an arrow B illustrated in FIG. 1.

The radioscopy apparatus 1 according to this embodiment comprises a main body portion 10. A plurality of wheels 11 are attached to the bottom of the main body portion 10, which makes it possible for the radioscopy apparatus 1 according to this embodiment to be moved. A support shaft 12 that is expanded and contracted in the z-axis direction of FIG. 1 is provided in an upper part of a housing of the main body portion 10 in FIG. 1. The bearing portion 9 is held in the upper part of the support shaft 12 so as to be movable in the direction of an arrow C.

With the above-described configuration, the radioscopy apparatus 1 according to this embodiment irradiates the subject H, who lies supine on the imaging table 14, with radiation from below the subject H, detects the radiation transmitted through the subject H with the radiation detector 5 of the imaging unit 3, and acquires a radiographic image of the subject H. Here, the C-arm 2 is movable in the direction of the arrow A, the direction of the arrow B, and the direction of the arrow C, and the radioscopy apparatus 1 is movable by the wheels 11. Therefore, the radioscopy apparatus 1 according to this embodiment can image a desired part of the subject H, who lies supine on the imaging table 14, in a desired direction.

In addition, in this embodiment, radiographic images are acquired by continuously irradiating the subject H with the radiation from the radiation source 6 during a treatment. In the following description, the acquired radiographic image is referred to as a fluoroscopic image.

A fluoroscopic image display device 20 according to a first embodiment is provided in the main body portion 10. FIG. 2 is a diagram illustrating a hardware configuration of the radioscopy apparatus and the fluoroscopic image display device according to this embodiment. In addition, in the following description, in some cases, it is assumed that the radioscopy apparatus and the fluoroscopic image display device are represented by the fluoroscopic image display device 20. As illustrated in FIG. 2, the fluoroscopic image display device 20 is a computer, such as a workstation, a server computer, or a personal computer, and comprises a central processing unit (CPU) 21, a non-volatile storage 23, and a memory 26 as a temporary storage area. Further, the fluoroscopic image display device 20 comprises a display 24, such as a liquid crystal display, an input device 25, such as a keyboard and a mouse, and an interface (I/F) 27. The CPU 21, the storage 23, the display 24, the input device 25, the memory 26, and the network I/F 27 are connected to a bus 28. In addition, the CPU 21 is an example of a processor according to the present disclosure.

The display 24 and the input device 25 are provided in an upper part of the main body portion 10 as illustrated in FIG. 1. The display 24 has a function of allowing a user, such as a radiology technician or a doctor who takes a radiographic image with the radioscopy apparatus 1, to input an instruction related to the capture of the radiographic image, a function of displaying the radiographic image acquired by imaging as a fluoroscopic image, and a function of providing the user with information related to the capture of the radiographic image. A touch panel display or the like in which the display 24 and the input device 25 are integrated may be used.

The storage 23 is implemented by, for example, a hard disk drive (HDD), a solid state drive (SSD), and a flash memory. The storage 23 as a storage medium stores a radioscopy program 22A installed in the radioscopy apparatus 1 and a fluoroscopic image display program 22B installed in the fluoroscopic image display device 20. The CPU 21 reads the radioscopy program 22A and the fluoroscopic image display program 22B from the storage 23, expands them into the memory 26, and executes the expanded radioscopy program 22A and fluoroscopic image display program 22B.

In addition, the radioscopy program 22A and the fluoroscopic image display program 22B are stored in a storage device of a server computer connected to a network or a network storage in a state in which they can be accessed from the outside and are downloaded and installed in a computer constituting the radioscopy apparatus 1 and the fluoroscopic image display device 20 as required. Alternatively, the programs are recorded on a recording medium, such as a digital versatile disc (DVD) or a compact disc read only memory (CD-ROM), and are distributed and installed in the computer constituting the radioscopy apparatus 1 and the fluoroscopic image display device 20 from the recording medium.

The I/F 27 has a function of communicating with a console and an external device (which are not illustrated), which perform overall control related to fluoroscopy by the radioscopy apparatus 1, in a wireless or wired manner. The radioscopy apparatus 1 according to this embodiment images the subject H on the basis of an imaging order received from the console through the I/F 27.

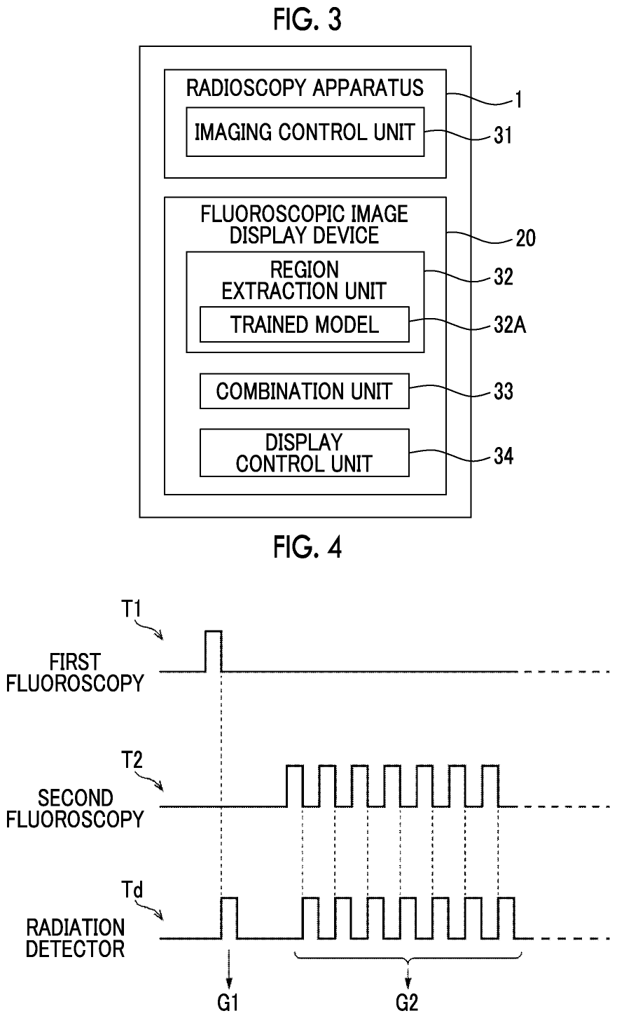

Next, the functional configurations of the radioscopy apparatus and the fluoroscopic image display device according to the first embodiment will be described. FIG. 3 is a diagram illustrating the functional configurations of the radioscopy apparatus and the fluoroscopic image display device according to the first embodiment. As illustrated in FIG. 3, the radioscopy apparatus 1 comprises an imaging control unit 31. Then, the CPU 21 executes the radioscopy program 22A to function as the imaging control unit 31.

Further, as illustrated in FIG. 3, the fluoroscopic image display device 20 comprises a region extraction unit 32, a combination unit 33, and a display control unit 34. Then, the CPU 21 executes the fluoroscopic image display program 22B to function as the region extraction unit 32, the combination unit 33, and the display control unit 34.

Here, in this embodiment, it is assumed that lumbar fusion is performed as a treatment for the subject H. Therefore, in this embodiment, it is assumed that the fluoroscopic images of the subject H are displayed as a moving image and the doctor performs a treatment of inserting a screw for fixing the lumbar spine into the lumbar spine while checking the depth and angle. In addition, the screw is an example of a treatment tool according to the present disclosure.

The imaging control unit 31 directs the radiation source 6 of the radiation emitting unit 4 to emit radiation on the basis of predetermined fluoroscopy conditions in order to acquire the fluoroscopic images as a moving image. Further, the imaging control unit 31 detects the radiation transmitted through the subject H with the radiation detector 5 of the imaging unit 3 according to the timing when the radiation source 6 emits the radiation and acquires the fluoroscopic image of the subject H.

Here, in this embodiment, the imaging control unit 31 controls the radiation source 6 and the radiation detector 5 such that first fluoroscopy is performed on the subject H before the treatment tool is inserted under predetermined first fluoroscopy conditions to acquire a first fluoroscopic image of the subject H. Further, the imaging control unit 31 controls the radiation source 6 and the radiation detector 5 such that second fluoroscopy is performed on the subject H after the treatment tool is inserted at a predetermined frame rate under second fluoroscopy conditions to sequentially acquire a second fluoroscopic image of the subject H.

The fluoroscopy conditions include at least one of a tube voltage or a tube current set for the radiation source 6. The first fluoroscopy conditions in a case in which the first fluoroscopy is performed include at least one of a first tube voltage or a first tube current. The second fluoroscopy conditions in a case in which the second fluoroscopy is performed include at least one of a second tube voltage or a second tube current.

As the tube voltage becomes higher, it is easier for radiation to be transmitted through the subject H. Therefore, the amount of radiation exposure to the subject H is reduced. Further, as the tube current becomes smaller, the radiation dose becomes smaller. Therefore, the amount of radiation exposure to the subject H is reduced. Therefore, the second tube voltage is higher than the first tube voltage, and the second tube current is smaller than the first tube current. For example, the first tube voltage can be set to 85 kV, the first tube current can be set to 10 mA, the second tube voltage can be set to 120 kV, and the second tube current can be set to 1 mA. However, the present disclosure is not limited thereto.

In addition, the first tube voltage and the second tube voltage may be equal to each other. In this case, the second tube current is smaller than the first tube current. Further, the first tube current and the second tube current may be equal to each other. In this case, the second tube voltage is higher than the first tube voltage. Therefore, the second fluoroscopic image acquired by the second fluoroscopy has a lower contrast than the first fluoroscopic image acquired by the first fluoroscopy.

Hereinafter, the timing when the radiation source 6 emits radiation and the timing when the radiation detector 5 detects the radiation in the first fluoroscopy and the second fluoroscopy will be described. FIG. 4 is a diagram illustrating the timing when the radiation source 6 emits radiation and the timing when the radiation detector 5 detects the radiation in the first embodiment. FIG. 4 illustrates a timing T1 when the radiation source 6 emits radiation in the first fluoroscopy, a timing T2 when the radiation source 6 emits radiation in the second fluoroscopy, and a timing Td when the radiation detector 5 detects the radiation transmitted through the subject H in the first fluoroscopy and the second fluoroscopy.

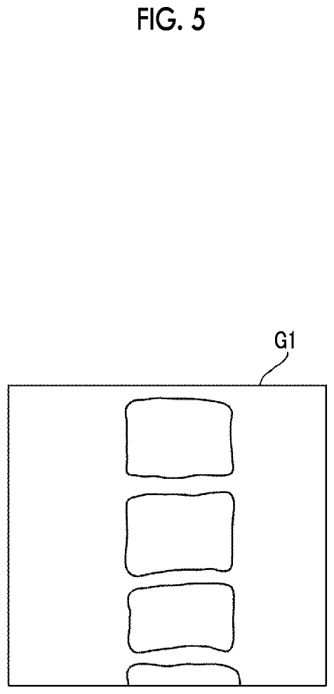

In a case in which the first fluoroscopy is performed, the imaging control unit 31 gives instructions to the radiation source 6 and the radiation detector 5 such that the radiation source 6 emits radiation under the first fluoroscopy conditions. Then, the radiation detector 5 detects the radiation transmitted through the subject H and outputs a first fluoroscopic image G1. FIG. 5 illustrates the first fluoroscopic image G1. In the first embodiment, the irradiation field stop 6A is set such that the first fluoroscopic image G1 including the vertebrae is acquired as illustrated in FIG. 5.

In a case in which the second fluoroscopy is performed after the first fluoroscopy, the imaging control unit 31 gives instructions to the radiation source 6 and the radiation detector 5 at a predetermined frame rate such that the radiation source 6 emits radiation at a predetermined frame rate under the second fluoroscopy conditions and the radiation detector 5 detects the radiation transmitted through the subject H and outputs second fluoroscopic images G2 at a predetermined frame rate. In addition, the second fluoroscopy may be started in response to an instruction from the operator through the input device 25 after the treatment is started.

In this embodiment, the predetermined frame rate is 25 to 60 fps, for example, 30 fps. Therefore, in this embodiment, the second fluoroscopic images G2 are acquired like a moving image. Further, in a case in which the second fluoroscopy is performed, the radiation source 6 may continuously emit radiation, and the radiation detector 5 may detect the radiation at a predetermined frame rate.

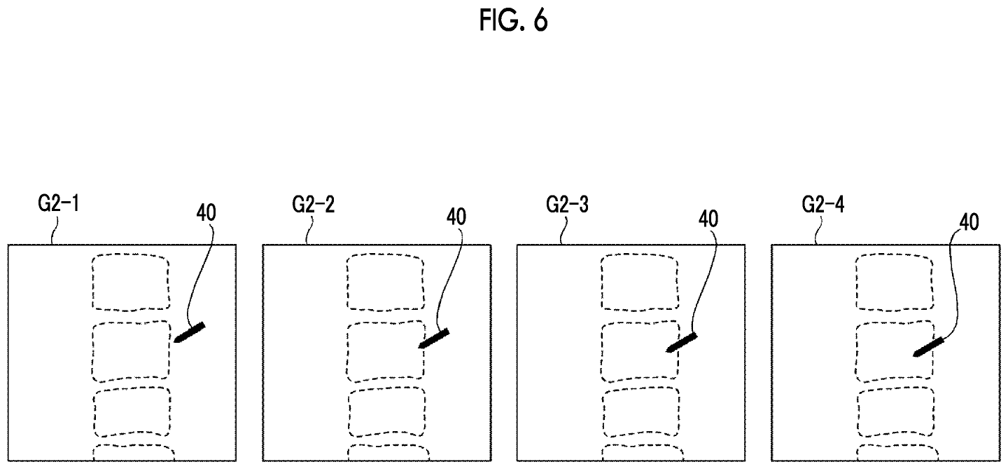

FIG. 6 illustrates the second fluoroscopic images. FIG. 6 illustrates four second fluoroscopic images G2-1, G2-2, G2-3, and G2-4 in chronological order. In FIG. 6, the contour of the vertebra is represented by a broken line, which shows that the second fluoroscopic image G2 has a lower contrast than the first fluoroscopic image G1. In addition, the second fluoroscopic image G2 includes a region 40 of a screw which is a treatment tool. Since the screw is made of metal, it is clearly shown even in the second fluoroscopic image G2 having a low contrast. Further, the second fluoroscopic image G2 corresponds to each frame of the moving image during the treatment. Therefore, in the second fluoroscopic images G2-1, G2-2, G2-3, and G2-4, a state in which the region 40 of the screw is gradually inserted into the vertebrae which is a target bone to be treated is shown in chronological order.

The region extraction unit 32 of the fluoroscopic image display device 20 according to the first embodiment extracts the region of the treatment tool from each of the second fluoroscopic images G2-i. In the first embodiment, the region 40 of the screw is extracted from each of the second fluoroscopic images G2-i illustrated in FIG. 6. For this purpose, the region extraction unit 32 has a trained model 32A that has been trained to extract the region 40 of the screw from each of the second fluoroscopic images G2-i.

The trained model 32A consists of a neural network that has been subjected to deep learning so as to extract the region of the screw included in the second fluoroscopic images G2-i. The trained model is generated by training the neural network using a large number of fluoroscopic images including the region of the screw as training data. Therefore, in a case in which the second fluoroscopic image G2 is input, the trained model 32A extracts the region of the screw included in the second fluoroscopic image G2.

Further, in addition to the neural network subjected to deep learning, any neural network subjected to machine learning, such as a support vector machine (SVM), a convolutional neural network (CNN), and a recurrent neural network (RNN), can be used for the trained model 32A.

Furthermore, the region extraction unit 32 is not limited to the configuration which extracts the region 40 of the screw using the trained model. For example, any method, such as a method using template matching, can be used as a method for extracting the region 40 of the screw.

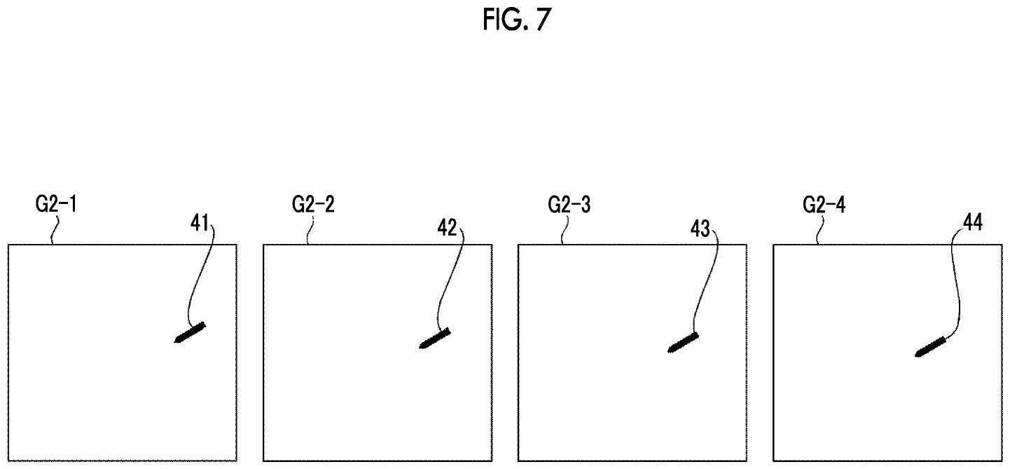

FIG. 7 is a diagram illustrating the extraction result of the region of the screw. As illustrated in FIG. 7, regions 41 to 44 of the screw are extracted from each of the second fluoroscopic images G2-1, G2-2, G2-3, and G2-4.

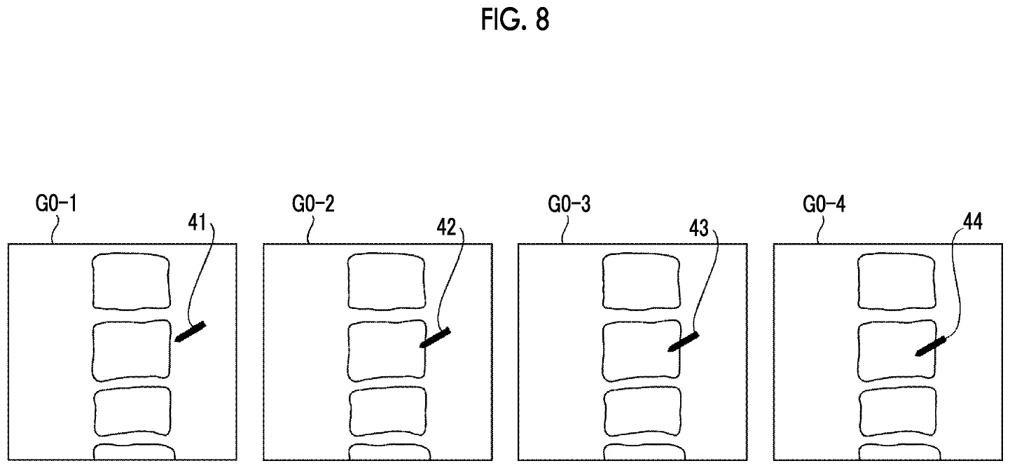

The combination unit 33 sequentially combines the regions 41 to 44 of the screw with the first fluoroscopic image G1 to sequentially derive composite fluoroscopic images at a predetermined frame rate. FIG. 8 is a diagram illustrating the composite fluoroscopic images that have been sequentially derived. FIG. 8 illustrates four composite fluoroscopic images G0-1, G0-2, G0-3, and G0-4 in chronological order. The four composite fluoroscopic images G0-1, G0-2, G0-3, and G0-4 include the regions 41 to 44 of the screw, respectively. Hereinafter, in some cases, it is assumed that the reference numerals of the composite fluoroscopic images G0-1, G0-2, G0-3, and G0-4 are represented by G0. Here, the first fluoroscopic image G1 has a higher contrast than the second fluoroscopic image G2. Therefore, in the composite fluoroscopic image G0, the region of the screw is superimposed on the high-quality first fluoroscopic image G1.

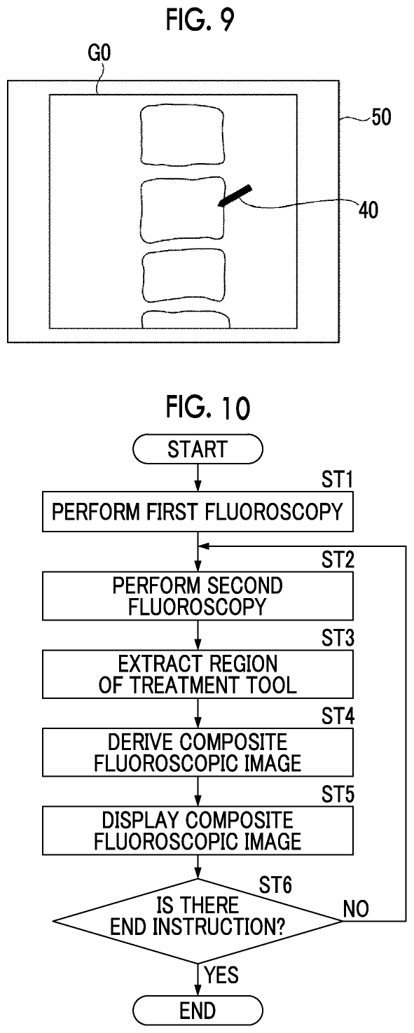

The display control unit 34 displays the composite fluoroscopic image G0 on the display 24. FIG. 9 is a diagram illustrating a composite fluoroscopic image display screen. As illustrated in FIG. 9, the composite fluoroscopic images G0 including the region 40 of the screw are displayed as a moving image at a predetermined frame rate on a display screen 50.

Next, a process performed in the first embodiment will be described. FIG. 10 is a flowchart illustrating the process performed in the first embodiment. The process is started in response to an imaging start instruction from the input device 25. First, the imaging control unit 31 of the radioscopy apparatus 1 controls the radiation source 6 and the radiation detector 5 such that the first fluoroscopy is performed under the first fluoroscopy conditions (Step ST1). Therefore, the first fluoroscopic image G1 of the subject H is acquired.

Then, in a case in which the screw, which is the treatment tool, is inserted into the subject H, the imaging control unit 31 controls the radiation source 6 and the radiation detector 5 such that the second fluoroscopy is performed under the second fluoroscopy conditions (Step ST2). Therefore, the second fluoroscopic image G2 is acquired.

Then, the region extraction unit 32 extracts the region of the treatment tool from the second fluoroscopic image G2 (Step ST3), and the combination unit 33 combines the region of the treatment tool with the first fluoroscopic image G1 to derive the composite fluoroscopic image G0 (Step ST4). Then, the display control unit 34 displays the composite fluoroscopic image G0 on the display 24 (Step ST5). Then, it is determined whether or not an end instruction is input (Step ST6). In a case in which the determination result in Step ST6 is “No”, the process returns to Step ST2. Then, the processes in Steps ST2 to ST6 are repeated. In a case in which the determination result in Step ST6 is “Yes”, the process ends.

As described above, in this embodiment, the region of the treatment tool extracted from the second fluoroscopic image G2 acquired at a predetermined frame rate under the second fluoroscopy conditions is sequentially combined with the first fluoroscopic image G1 acquired under the first fluoroscopy conditions to derive the composite fluoroscopic image G0. Here, the second tube voltage included in the second fluoroscopy conditions is higher than the first tube voltage, and the second tube current is smaller than the first tube current. Therefore, the radiation dose emitted to the subject H in a case in which the second fluoroscopic image G2 is acquired is smaller than the radiation dose in a case in which the first fluoroscopic image G1 is acquired. As a result, it is possible to reduce the amount of radiation exposure to the subject H in a case in which the fluoroscopic image of the subject H is captured during the treatment.

Next, a second embodiment of the present disclosure will be described. In addition, since the functional configurations of a radioscopy apparatus and a fluoroscopic image display device according to the second embodiment are the same as the functional configurations of the radioscopy apparatus and the fluoroscopic image display device according to the first embodiment illustrated in FIG. 3, the detailed description of the functional configurations of the apparatus and the device will not be repeated here.

The radioscopy apparatus and the fluoroscopic image display device according to the second embodiment differ from those according to the first embodiment in that the region of the treatment tool is detected from one second fluoroscopic image G2, the irradiation field stop 6A is set such that a range, which includes the detected treatment tool and is narrower than that in a case in which the first fluoroscopic image G1 is acquired, is irradiated with radiation, radiation is regulated by the set irradiation field stop, and the subject H is irradiated with the radiation to perform the second fluoroscopy after the one second fluoroscopic image G2 is acquired.

In the second embodiment, the imaging control unit 31 sets the irradiation field stop 6A. Here, a position on the second fluoroscopic image G2 corresponds to a position on the radiation detector 5. Therefore, first, the imaging control unit 31 specifies the region of the treatment tool detected from one second fluoroscopic image G2 by the region extraction unit 32 on the radiation detector 5. In addition, the one second fluoroscopic image G2 may be the second fluoroscopic image G2 acquired first by the second fluoroscopy. However, the present disclosure is not limited thereto.

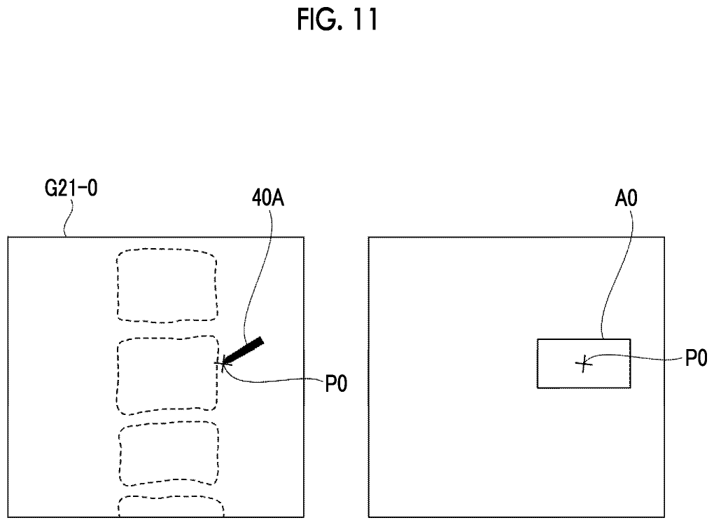

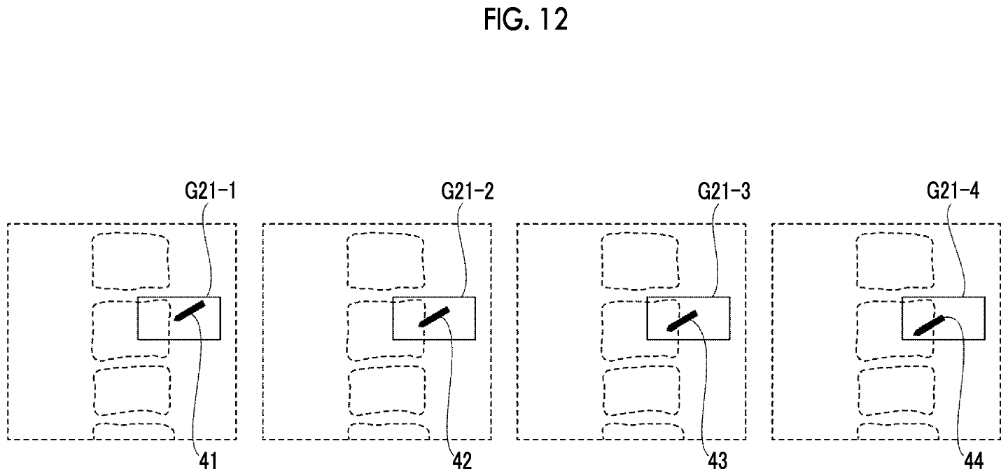

Then, the imaging control unit 31 sets the center position of the aperture of the irradiation field stop 6A accommodated in the radiation emitting unit 4 and the size of the aperture such that the range, which includes the region of the treatment tool detected from the second fluoroscopic image G2 and is narrower than that in a case in which the first fluoroscopic image G1 is acquired, in the radiation detector 5 is irradiated with radiation. FIG. 11 is a diagram illustrating the setting of the irradiation field stop 6A in the second embodiment. As illustrated in FIG. 11, the imaging control unit 31 specifies a tip position P0 of the treatment tool in a region 40A of the screw which is the treatment tool detected from one second fluoroscopic image (denoted by G21-0) by the region extraction unit 32. The imaging control unit 31 sets a predetermined region, which is smaller than the first fluoroscopic image G1 and has the tip position P0 as the center, as an irradiation field region A0. In addition, the size of the irradiation field region A0 may be preset according to the size of the treatment tool, a position where the treatment tool is inserted, or a moving direction of the treatment tool. Further, the irradiation field region A0 may be set such that the irradiation range of radiation with respect to a traveling direction of the screw, which is the treatment tool, is widened. Then, the imaging control unit 31 sets the center position of the aperture of the irradiation field stop 6A accommodated in the radiation emitting unit 4 and the size of the aperture such that the irradiation field region A0 is irradiated with radiation.

In the second embodiment, after the irradiation field stop 6A is set, the imaging control unit 31 controls the radiation source 6 and the radiation detector 5 to continue the second fluoroscopy. Therefore, the subject H is irradiated with the radiation regulated by the set irradiation field stop, and the second fluoroscopic image G2 is acquired. In addition, the irradiation field stop 6A may be manually set by the operator.

FIG. 12 is a diagram illustrating the second fluoroscopic images acquired in the second embodiment. As illustrated in FIG. 12, second fluoroscopic images G21-1 to G21-4 acquired following the second fluoroscopic image G21-0 in the second embodiment are regions that include the regions 41 to 44 of the screw, which is the treatment tool, in the subject H and have a smaller size than the first fluoroscopic image G1.

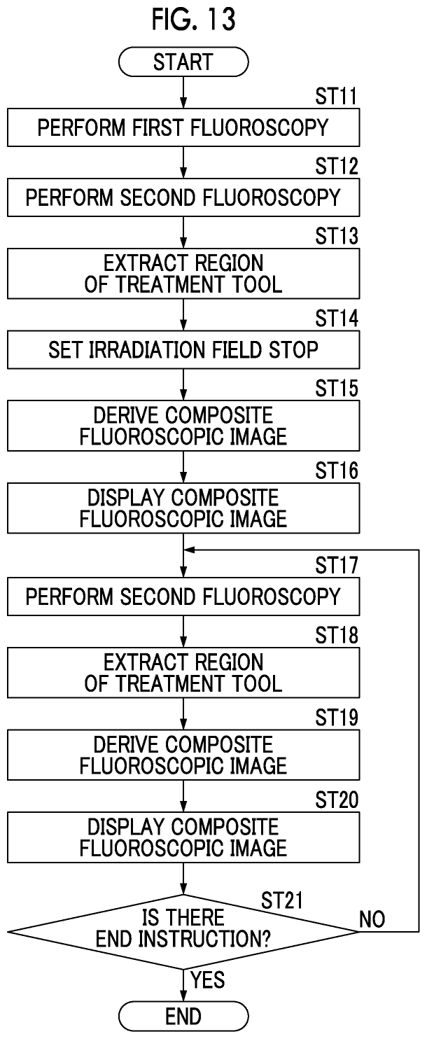

Next, a process performed in the second embodiment will be described. FIG. 13 is a flowchart illustrating the process performed in the second embodiment. The process is started in response to an imaging start instruction from the input device 25. First, the imaging control unit 31 of the radioscopy apparatus 1 controls the radiation source 6 and the radiation detector 5 such that the first fluoroscopy is performed under the first fluoroscopy conditions (Step ST11). Therefore, the first fluoroscopic image G1 of the subject H is acquired.

Then, in a case in which the screw, which is the treatment tool, is inserted into the subject H, the imaging control unit 31 controls the radiation source 6 and the radiation detector 5 such that the second fluoroscopy is performed under the second fluoroscopy conditions (Step ST12). Therefore, the second fluoroscopic image G2 is acquired. Then, the region extraction unit 32 extracts the region of the treatment tool from the first second fluoroscopic image G2 (Step ST13), and the imaging control unit 31 sets the irradiation field stop 6A such that the range, which includes the treatment tool and is narrower than that in a case in which the first fluoroscopic image G1 is acquired, is irradiated with radiation (Step ST14). Further, the combination unit 33 combines the region of the treatment tool with the first fluoroscopic image G1 to derive the composite fluoroscopic image G0 (Step ST15), and the display control unit 34 displays the composite fluoroscopic image G0 on the display 24 (Step ST16). In addition, the processes in Steps ST15 and ST16 may be performed before Step ST14 or may be performed in parallel to Step ST14.

Then, the imaging control unit 31 controls the radiation source 6 and the radiation detector 5 such that the second fluoroscopy is performed under the second fluoroscopy conditions (Step ST17). Therefore, the second fluoroscopic image G2 having a narrower range than the first fluoroscopic image G1 is continuously acquired.

Then, the region extraction unit 32 extracts the region of the treatment tool from the second fluoroscopic image G2 (Step ST18), and the combination unit 33 combines the region of the treatment tool with the first fluoroscopic image G1 to derive the composite fluoroscopic image G0 (Step ST19). Then, the display control unit 34 displays the composite fluoroscopic image G0 on the display 24 (Step ST20). Then, it is determined whether or not an end instruction is input (Step ST21). In a case in which the determination result in Step ST21 is “No”, the process returns to Step ST17. Then, the processes in Steps ST17 to ST21 are repeated. In a case in which the determination result in Step ST21 is “Yes”, the process ends.

As described above, in the second embodiment, in a case in which the second fluoroscopy is performed, only the range that includes the treatment tool and is narrower than that in a case in which the first fluoroscopic image G1 is acquired is irradiated with radiation. Therefore, it is possible to further reduce the amount of radiation exposure to the subject H during the second fluoroscopy.

In addition, the treatment tool is moved during the treatment. Therefore, in the second embodiment, the region of the treatment tool may be detected again from the second fluoroscopic image G2 during the treatment, and the irradiation field region A0 may be reset.

Next, a third embodiment of the present disclosure will be described. Further, since the functional configurations of a radioscopy apparatus and a fluoroscopic image display device according to the third embodiment are the same as the functional configurations of the radioscopy apparatus and the fluoroscopic image display device according to the first embodiment illustrated in FIG. 3, the detailed description of the functional configurations of the apparatus and the device will not be repeated here.

The radioscopy apparatus and the fluoroscopic image display device according to the third embodiment differ from those according to the first embodiment in that, after the first fluoroscopic image G1 is acquired, the imaging control unit 31 controls the radiation source 6 and the radiation detector 5 such that third fluoroscopy which sequentially acquires a third fluoroscopic image at a frame rate higher than the frame rate at which the second fluoroscopy is performed is further performed under the first fluoroscopy conditions.

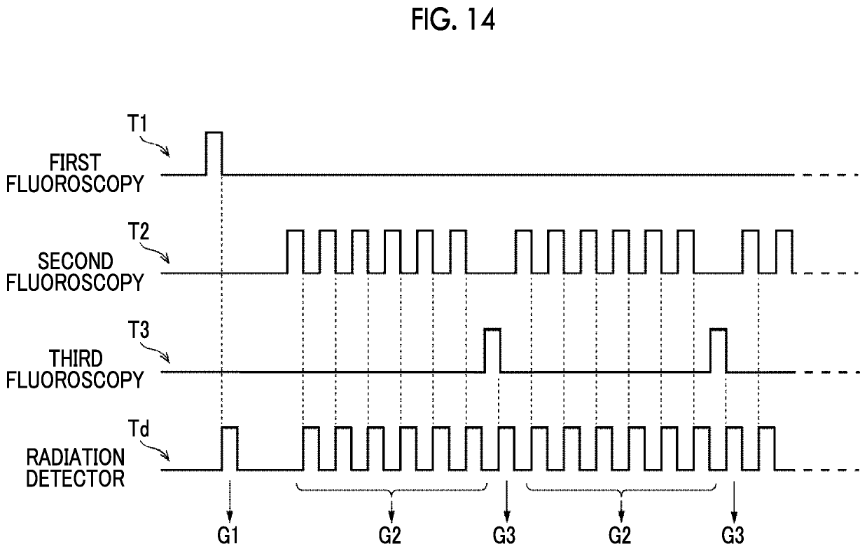

Hereinafter, the timing when the radiation source 6 emits radiation and the timing when the radiation detector 5 detects the first to third fluoroscopic images in the first fluoroscopy to the third fluoroscopy in the third embodiment will be described. FIG. 14 is a diagram illustrating the timing when the radiation source 6 emits radiation and the timing when the radiation detector 5 detects the radiation in the third embodiment.

In addition, FIG. 14 illustrates a timing T1 when the radiation source 6 emits radiation in the first fluoroscopy, a timing T2 when the radiation source 6 emits radiation in the second fluoroscopy, a timing T3 when the radiation source 6 emits radiation in the third fluoroscopy, and a timing Td when the radiation detector 5 detects the radiation transmitted through the subject H in the first fluoroscopy to the third fluoroscopy.

In the third embodiment, the first fluoroscopy is performed in the same manner as that in the first and second embodiments to acquire the first fluoroscopic image G1.

In a case in which the second fluoroscopy is performed after the first fluoroscopy, the imaging control unit 31 gives instructions to the radiation source 6 and the radiation detector 5 at a predetermined frame rate such that the radiation source 6 emits radiation at a predetermined frame rate under the second fluoroscopy conditions and the radiation detector 5 detects the radiation transmitted through the subject H and outputs the second fluoroscopic image G2 at a predetermined frame rate. In addition, the frame rate in a case in which the second fluoroscopy is performed is equal to that in the second fluoroscopy according to the first embodiment. The frame rate in a case in which the second fluoroscopy is performed in the third embodiment is referred to as a first frame rate.

Further, in the third embodiment, in a case in which the second fluoroscopy is performed, the radiation source 6 may continuously emit radiation, and the radiation detector 5 may detect the radiation at the first frame rate.

Then, in a case in which the third fluoroscopy is performed, the imaging control unit 31 gives instructions to the radiation source 6 and the radiation detector 5 at a second frame rate lower than the first frame rate such that the radiation source 6 emits radiation at the second frame rate under the first fluoroscopy conditions and the radiation detector 5 detects the radiation transmitted through the subject H and outputs a third fluoroscopic image G3 at the second frame rate. In addition, a first third fluoroscopy operation is performed at a timing when the time corresponding to the second frame rate has elapsed since the start of the first second fluoroscopy. Further, the second fluoroscopy is not performed at the timing when the third radioscopy is performed.

In the third embodiment, the second frame rate may be lower than the first frame rate. For example, in FIG. 14, the second frame rate is 1 fps which is 1/6 of the first frame rate. However, the present disclosure is not limited thereto.

In the third embodiment, the combination unit 33 sequentially combines the region of the screw extracted by the region extraction unit 32 with the first fluoroscopic image G1 to sequentially derive the composite fluoroscopic image G0 at a predetermined frame rate (that is, the first frame rate) from the first fluoroscopy to the third fluoroscopy as in the first embodiment.

In a case in which the third fluoroscopy is performed, the combination unit 33 sequentially combines the region of the screw extracted by the region extraction unit 32 with the third fluoroscopic image G3 instead of the first fluoroscopic image G1 to sequentially derive other composite fluoroscopic images G10 at the second frame rate. Then, in a case in which the third fluoroscopy is further performed at a timing after the lapse of the time corresponding to the second frame rate, the combination unit 33 sequentially combines the region of the screw extracted by the region extraction unit 32 with a new third fluoroscopic image G3 to sequentially derive other composite fluoroscopic images G10 at the second frame rate.

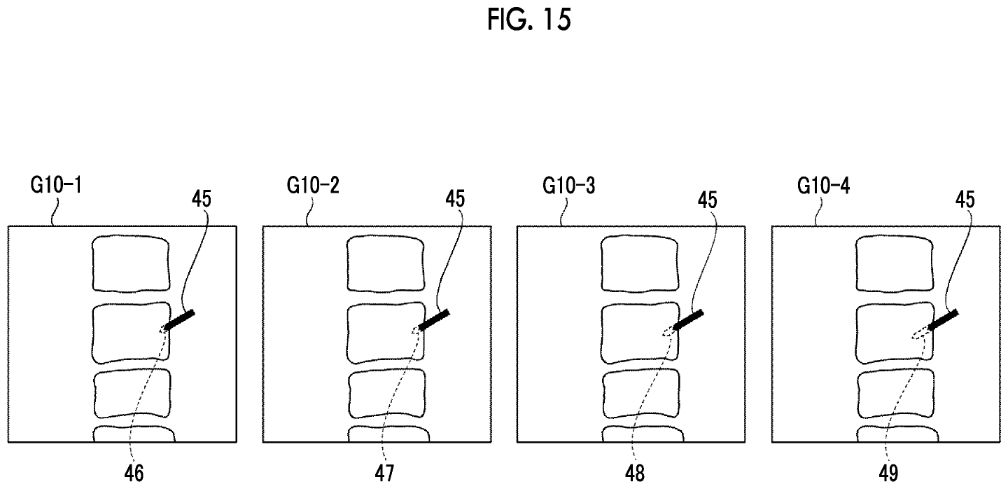

FIG. 15 is a diagram illustrating other composite fluoroscopic images that have been derived sequentially. FIG. 15 illustrates four other composite fluoroscopic images G10-1, G10-2, G10-3, and G10-4 in chronological order. Hereinafter, in some cases, it is assumed that the reference numerals of other composite fluoroscopic images G10-1, G10-2, G10-3, and G10-4 are represented by G10. Here, the third fluoroscopic image G3 has a higher contrast than the second fluoroscopic image G2 similarly to the first fluoroscopic image G1. Therefore, in other composite fluoroscopic images G10, the region of the screw is superimposed on the high-quality fluoroscopic image. In addition, since the third fluoroscopic image G3 is acquired after the treatment is started, it includes the region 45 of the screw. Therefore, in other composite fluoroscopic images G10-1, G10-2, G10-3, and G10-4, regions 46 to 49 of the screw extracted in the second fluoroscopic image G2 gradually extend from the tip of the region 45 of the screw included in the third fluoroscopic image G3.

Further, in FIG. 15, for the sake of explanation, the regions 46 to 49 of the screw extracted in the second fluoroscopic image G2 are represented by broken lines. However, in practice, the regions 46 to 49 of the screw have substantially the same contrast as the region 45 of the screw extracted in the third fluoroscopic image G3.

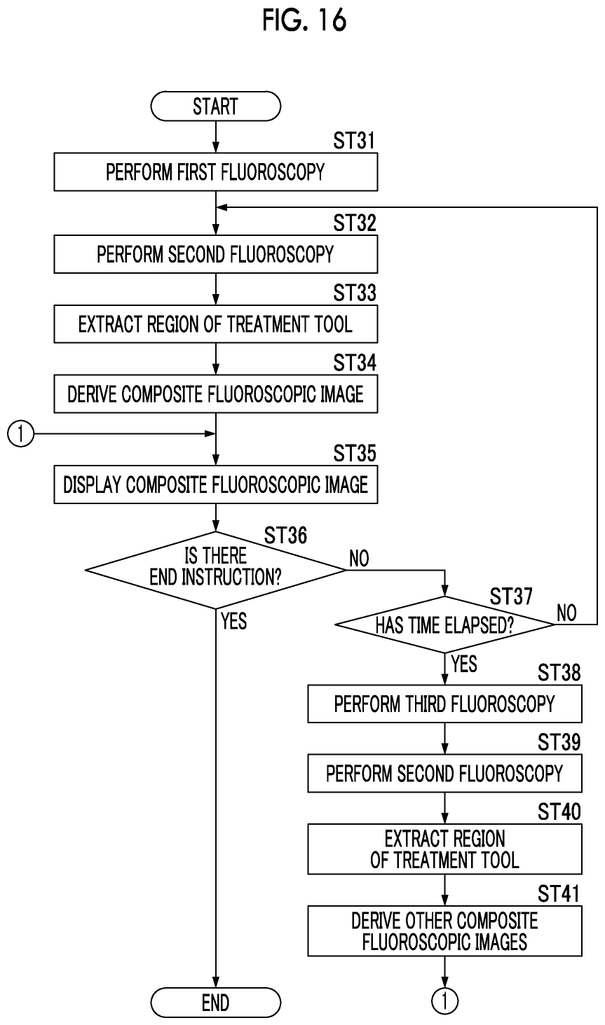

Next, a process performed in the third embodiment will be described. FIG. 16 is a flowchart illustrating the process performed in the third embodiment. The process is started in response to an imaging start instruction from the input device 25. First, the imaging control unit 31 of the radioscopy apparatus 1 controls the radiation source 6 and the radiation detector 5 such that the first fluoroscopy is performed under the first fluoroscopy conditions (Step ST31). Therefore, the first fluoroscopic image G1 of the subject H is acquired.

Then, in a case in which the screw, which is the treatment tool, is inserted into the subject H, the imaging control unit 31 controls the radiation source 6 and the radiation detector 5 such that the second fluoroscopy is performed under the second fluoroscopy conditions (Step ST32). Therefore, the second fluoroscopic image G2 is acquired.

Then, the region extraction unit 32 extracts the region of the treatment tool from the second fluoroscopic image G2 (Step ST33), and the combination unit 33 combines the region of the treatment tool with the first fluoroscopic image G1 to derive the composite fluoroscopic image G0 (Step ST34). Then, the display control unit 34 displays the composite fluoroscopic image G0 on the display 24 (Step ST35). Then, it is determined whether or not an end instruction is input (Step ST36). In a case in which the determination result in Step ST36 is “Yes”, the process ends. In a case in which the determination result in Step ST36 is “No”, the imaging control unit 31 determines whether or not the time corresponding to the second frame rate has elapsed (lapse of time: Step ST37).

In a case in which the determination result in Step ST37 is “No”, the process returns to Step ST32. Then, the processes in Step ST32 to Step ST37 are repeated. In a case in which the determination result in Step ST37 is “Yes”, the imaging control unit 31 controls the radiation source 6 and the radiation detector 5 such that the third fluoroscopy is performed under the first fluoroscopy conditions (Step ST38). Therefore, the third fluoroscopic image G3 is acquired. Then, the imaging control unit 31 controls the radiation source 6 and the radiation detector 5 such that the second fluoroscopy is performed under the second fluoroscopy conditions (Step ST39). Therefore, the second fluoroscopic image G2 is acquired.

Then, the region extraction unit 32 extracts the region of the treatment tool from the second fluoroscopic image G2 (Step ST40), and the combination unit 33 combines the region of the treatment tool with the third fluoroscopic image G3 to derive other composite fluoroscopic images G10 (Step ST41). Then, the process returns to Step ST35. In Step ST35, the display control unit 34 displays other composite fluoroscopic images G10 on the display 24.

As described above, in the third embodiment, after the first fluoroscopic image G1 is acquired, the third fluoroscopy which sequentially acquires the third fluoroscopic image G3 at a frame rate lower than the frame rate at which the second fluoroscopy is performed is further performed under the first fluoroscopy conditions, and the region of the treatment tool extracted from the second fluoroscopic image G2 is combined with the third fluoroscopic image G3 to derive other composite fluoroscopic images G10.

Therefore, a fluoroscopic image which is the background of the region of the treatment tool is reset whenever the time corresponding to the second frame rate elapses. Therefore, even in a case in which the body movement of the subject H occurs during the treatment, it is possible to display other composite fluoroscopic images G10 in which the deviation between the image which is the background of the region of the treatment tool and the region of the treatment tool has been reduced. As a result, according to the third embodiment, it is possible to accurately check the progress of the treatment.

In addition, in the third embodiment, as in the second embodiment, radiation may be regulated by the irradiation field stop, and the subject H may be irradiated with the radiation to perform the second fluoroscopy and the third radioscopy.

Further, in each of the above-described embodiments, the radioscopy apparatus and the fluoroscopic image display device according to the present disclosure are applied to acquire the fluoroscopic images in a case in which the lumbar fusion is performed. However, the present invention is not limited thereto. For example, the radioscopy apparatus and the fluoroscopic image display device according to the present disclosure may be applied to acquire fluoroscopic images in a case in which a catheter treatment for the abdominal aneurysm of the subject H is performed. In this case, the treatment tool is a stent that is placed in the artery and a guide wire for guiding the stent. The operator performs a procedure of placing the stent at a desired position in the artery with the guide wire while viewing the fluoroscopic image displayed on the display 24. In addition to this, the technology of the present disclosure can be applied to a case in which any treatment is performed as long as it uses a fluoroscopic image.

Further, in each of the above-described embodiments, the radiation is not particularly limited. For example, α-rays or γ-rays other than X-rays can be applied.

Furthermore, in the above-described embodiments, for example, the following various processors can be used as a hardware structure of processing units performing various processes, such as the imaging control unit 31, the region extraction unit 32, the combination unit 33, and the display control unit 34. The various processors include, for example, a CPU which is a general-purpose processor executing software (program) to function as various processing units as described above, a programmable logic device (PLD), such as a field programmable gate array (FPGA), which is a processor whose circuit configuration can be changed after manufacture, and a dedicated electric circuit, such as an application specific integrated circuit (ASIC), which is a processor having a dedicated circuit configuration designed to perform a specific process.

One processing unit may be configured by one of the various processors or a combination of two or more processors of the same type or different types (for example, a combination of a plurality of FPGAs or a combination of a CPU and an FPGA). Further, a plurality of processing units may be configured by one processor.

A first example of the configuration in which a plurality of processing units are configured by one processor is an aspect in which one processor is configured by a combination of one or more CPUs and software and functions as a plurality of processing units. A representative example of this aspect is a client computer or a server computer. A second example of the configuration is an aspect in which a processor that implements the functions of the entire system including a plurality of processing units using one integrated circuit (IC) chip is used. A representative example of this aspect is a system-on-chip (SoC). As such, various processing units are configured using one or more of the various processors as the hardware structure.

In addition, specifically, an electric circuit (circuitry) obtained by combining circuit elements, such as semiconductor elements, can be used as the hardware structure of the various processors.

What is claimed is:

1. A radioscopy apparatus comprising:
a radiation source that irradiates a subject with radiation;
a radiation detector that detects the radiation transmitted through the subject to generate a fluoroscopic image of the subject; and
at least one processor,
wherein the processor controls the radiation source and the radiation detector such that first fluoroscopy is performed on the subject before a treatment tool is inserted under first fluoroscopy conditions including at least one of a predetermined first tube voltage or a predetermined first tube current to acquire a first fluoroscopic image of the subject, and controls the radiation source and the radiation detector such that second fluoroscopy is performed at a predetermined frame rate on the subject after the treatment tool is inserted under second fluoroscopy conditions including at least one of a second tube voltage higher than the first tube voltage or a second tube current smaller than the first tube current to sequentially acquire a plurality of second fluoroscopic images of the subject.

2. The radioscopy apparatus according to claim 1, further comprising:
an irradiation field stop that regulates a range in which the subject is irradiated with the radiation,
wherein the processor detects the treatment tool from one of the second fluoroscopic images, sets the irradiation field stop such that a range, which includes the detected treatment tool and is narrower than that in a case in which the first fluoroscopic image is acquired, is irradiated with the radiation, and regulates the radiation with the set irradiation field stop and irradiates the subject with the radiation to perform the second fluoroscopy after the one second fluoroscopic image is acquired.

3. The radioscopy apparatus according to claim 1,
wherein, after acquiring the first fluoroscopic image, the processor controls the radiation source and the radiation detector such that third fluoroscopy which sequentially acquires a third fluoroscopic image at a frame rate lower than the predetermined frame rate is further performed under the first fluoroscopy conditions.

4. A fluoroscopic image display device comprising at least one processor,
wherein the processor acquires the first fluoroscopic image acquired by the radioscopy apparatus according to claim 1, sequentially acquires the second fluoroscopic images acquired by the radioscopy apparatus according to claim 1, sequentially extracts a region of the treatment tool from each of the second fluoroscopic images, sequentially combines the region of the treatment tool with the first fluoroscopic image to sequentially derive a composite fluoroscopic image at the predetermined frame rate, and sequentially displays the composite fluoroscopic image.

5. A fluoroscopic image display device comprising at least one processor,
wherein the processor acquires the first fluoroscopic image acquired by the radioscopy apparatus according to claim 3, sequentially acquires the second fluoroscopic images acquired by the radioscopy apparatus according to claim 3, sequentially extracts a region of the treatment tool from each of the second fluoroscopic images, sequentially combines the region of the treatment tool with the first fluoroscopic image to sequentially derive a composite fluoroscopic image at the predetermined frame rate, displays the composite fluoroscopic image, sequentially acquires the third fluoroscopic image acquired by the radioscopy apparatus according to claim 3, sequentially combines the region of the treatment tool extracted from the second fluoroscopic image acquired until a next third fluoroscopic image is acquired with the sequentially acquired third fluoroscopic images to sequentially derive other composite fluoroscopic images, and sequentially displays the other composite fluoroscopic images instead of the composite fluoroscopic image.

6. A radioscopy method in a radioscopy apparatus including a radiation source that irradiates a subject with radiation and a radiation detector that detects the radiation transmitted through the subject to generate a fluoroscopic image of the subject, the radioscopy method comprising:
controlling the radiation source and the radiation detector such that first fluoroscopy is performed on the subject before a treatment tool is inserted under first fluoroscopy conditions including at least one of a predetermined first tube voltage or a predetermined first tube current to acquire a first fluoroscopic image of the subject; and
controlling the radiation source and the radiation detector such that second fluoroscopy is performed at a predetermined frame rate on the subject after the treatment tool is inserted under second fluoroscopy conditions including at least one of a second tube voltage higher than the first tube voltage or a second tube current smaller than the first tube current to sequentially acquire a plurality of second fluoroscopic images of the subject.

7. A fluoroscopic image display method comprising:
acquiring the first fluoroscopic image acquired by the radioscopy apparatus according to claim 1;
sequentially acquiring the second fluoroscopic images acquired by the radioscopy apparatus according to claim 1;
sequentially extracting a region of the treatment tool from each of the second fluoroscopic images;
sequentially combining the region of the treatment tool with the first fluoroscopic image to sequentially derive a composite fluoroscopic image at the predetermined frame rate; and
sequentially displaying the composite fluoroscopic image.

8. A fluoroscopic image display method comprising:
acquiring the first fluoroscopic image acquired by the radioscopy apparatus according to claim 3;
sequentially acquiring the second fluoroscopic images acquired by the radioscopy apparatus according to claim 3;
sequentially extracting a region of the treatment tool from each of the second fluoroscopic images;
sequentially combining the region of the treatment tool with the first fluoroscopic image to sequentially derive a composite fluoroscopic image at the predetermined frame rate;
displaying the composite fluoroscopic image;
sequentially acquiring the third fluoroscopic image acquired by the radioscopy apparatus according to claim 3;
sequentially combining the region of the treatment tool extracted from the second fluoroscopic image acquired until a next third fluoroscopic image is acquired with the sequentially acquired third fluoroscopic images to sequentially derive other composite fluoroscopic images; and
sequentially displaying the other composite fluoroscopic images instead of the composite fluoroscopic image.

9. A radioscopy program that causes a computer to perform a radioscopy method in a radioscopy apparatus including a radiation source that irradiates a subject with radiation and a radiation detector that detects the radiation transmitted through the subject to generate a fluoroscopic image of the subject, the radioscopy program causing the computer to execute:
a procedure of controlling the radiation source and the radiation detector such that first fluoroscopy is performed on the subject before a treatment tool is inserted under first fluoroscopy conditions including at least one of a predetermined first tube voltage or a predetermined first tube current to acquire a first fluoroscopic image of the subject; and
a procedure of controlling the radiation source and the radiation detector such that second fluoroscopy is performed at a predetermined frame rate on the subject after the treatment tool is inserted under second fluoroscopy conditions including at least one of a second tube voltage higher than the first tube voltage or a second tube current smaller than the first tube current to sequentially acquire a plurality of second fluoroscopic images of the subject.

10. A non-transitory computer-readable storage medium that stores a fluoroscopic image display program that causes a computer to execute:
a procedure of acquiring the first fluoroscopic image acquired by the radioscopy apparatus according to claim 1;
a procedure of sequentially acquiring the second fluoroscopic images acquired by the radioscopy apparatus according to claim 1;
a procedure of sequentially extracting a region of the treatment tool from each of the second fluoroscopic images;
a procedure of sequentially combining the region of the treatment tool with the first fluoroscopic image to sequentially derive a composite fluoroscopic image at the predetermined frame rate; and
a procedure of sequentially displaying the composite fluoroscopic image.

11. A non-transitory computer-readable storage medium that stores a fluoroscopic image display program that causes a computer to execute:
a procedure of acquiring the first fluoroscopic image acquired by the radioscopy apparatus according to claim 3;
a procedure of sequentially acquiring the second fluoroscopic images acquired by the radioscopy apparatus according to claim 3;
a procedure of sequentially extracting a region of the treatment tool from each of the second fluoroscopic images;
a procedure of sequentially combining the region of the treatment tool with the first fluoroscopic image to sequentially derive a composite fluoroscopic image at the predetermined frame rate;
a procedure of displaying the composite fluoroscopic image;
a procedure of sequentially acquiring the third fluoroscopic image acquired by the radioscopy apparatus according to claim 3;
a procedure of sequentially combining the region of the treatment tool extracted from the second fluoroscopic image acquired until a next third fluoroscopic image is acquired with the sequentially acquired third fluoroscopic images to sequentially derive other composite fluoroscopic images; and
a procedure of sequentially displaying the other composite fluoroscopic images instead of the composite fluoroscopic image.
